(19) United States
(12) Patent Application Publication
(10) Pub. No.: US 2016/0312274 A1
(43) Pub. Date: Oct. 27, 2016

(54) METHODS AND SYSTEMS FOR VOLUME VARIATION MODELING IN DIGITAL PCR

(71) Applicant: LIFE TECHNOLOGIES CORPORATION, Carlsbad, CA (US)

(72) Inventors: Nivedita Sumi Majumdar, San Bruno, CA (US); Swapnonil Banerjee, San Bruno, CA (US)

(21) Appl. No.: 15/136,774

(22) Filed: Apr. 22, 2016

Related U.S. Application Data
(60) Provisional application No. 62/152,542, filed on Apr. 24, 2015.
(60) Provisional application No. 62/193,932, filed on Jul. 17, 2015.

(51) Int. Cl. C12Q 1/68; G06F 17/18

(52) U.S. Cl. CPC C12Q 1/6851; G06F 17/18

(57) ABSTRACT

A method for performing digital polymerase chain reaction (dPCR) is provided. The method includes partitioning a biological sample volume including a plurality of target nucleic acids into a plurality of partitions, where at least one partition includes at least one target nucleic acid. The method further includes determining a model for volume variation of the plurality of partitions and determining a number of partitions including at least one target nucleic acid. The method includes generating a concentration of target nucleic acids in the biological sample based on the model for volume variation and the fraction of partitions including at least one target nucleic acid.

### CROSS-REFERENCE TO RELATED APPLICATIONS

This application claims the benefit of priority of U.S. Provisional Patent Application No. 62/152,542, filed on Apr. 24, 2015, and U.S. Provisional Patent Application No. 62/193,932, filed on Jul. 17, 2015, which are both incorporated herein in their entireties by reference.

### BACKGROUND

The great promise of digital PCR is the potential for unparalleled precision enabling accurate measurements for genetic quantification. Target nucleic acid molecules of a sample requiring quantification are distributed evenly on a digital PCR consumable with many partitions and subjected to a PCR reaction. Partitions with template show amplification of the target nucleic acid and partitions lacking template do not show amplification. The observations are typically fitted with the Poisson model to predict the number of molecules present in the sample under measurement.

A basic assumption of the Poisson model is that molecules are equally likely to be present in any given partition, implying that the partitions are of equal size. For microfluidics, functioning at sub nanoliter volumes, this assumption may easily be violated. Recently, discussions of the detrimental effects of partition size variation on digital PCR quantification results, particularly at high concentrations, have come to light.

### SUMMARY

A method for performing digital polymerase chain reaction (dPCR) is provided. The method includes partitioning a biological sample volume including a plurality of target nucleic acids into a plurality of partitions, where at least one partition includes at least one target nucleic acid. The method further includes determining a model for volume variation of the plurality of partitions and determining a number of partitions including at least one target nucleic acid. The method includes generating a concentration of target nucleic acids in the biological sample based on the model for volume variation and the fraction of partitions including at least one target nucleic acid.

A system for performing digital polymerase chain reaction (dPCR) is provided. The system includes a device configured to partition a biological sample volume including a plurality of target nucleic acids into a plurality of partitions, where at least one partition includes at least one target nucleic acid. The system further includes a memory, and a processor configured to determine a number of partitions including at least one target nucleic acid, and generate a concentration of target nucleic acids in the biological sample based on a model for volume variation and the fraction of partitions including at least one target nucleic acid.

In various embodiments, the concentration of target nucleic acids in the biological sample is generated by using the equation:

$$C = \frac{{v}_{0} - \sqrt{{v}_{0}^{2} + 2 {σ}^{2} ln P ( neg )}}{{σ}^{2}} ,$$

when using the Variable λ Approximation Model
Or by implicit solution of the equation, when using the Variable λ Full Fidelity model

$$P ( neg ) = \frac{erfc [ - \frac{1}{\sqrt{2}} ( \frac{{v}_{0}}{σ} - C σ ) ]}{erfc [ - \frac{1}{\sqrt{2}} ( \frac{{v}_{0}}{σ} ) ]} {e}^{- {Cv}_{0} + \frac{1}{2} {σ}^{2} {C}^{2}}$$

### DESCRIPTION OF THE FIGURES

FIG. 1 illustrates that volume variation impacts higher concentration more significantly than lower concentration according to various embodiments described herein;

FIG. 2A-2D illustrates show increasingly higher partition size non-uniformities created by assuming a standard deviation of 15%, 25%, 35% and 50% of the mean volumes, respectively according to various embodiments described herein;

FIG. 3 illustrates an example of quantification results and prediction error percent using a Poisson calculation and the volume variation method according to various embodiment described herein;

FIG. 4 illustrates an exemplary computing system for implementing various embodiments described herein; and

FIG. 5 illustrates an exemplary distributed network system according to various embodiments described herein.

### DETAILED DESCRIPTION

To provide a more thorough understanding of various embodiments, the following description sets forth numerous specific details, such as specific configurations, parameters, examples, and the like. It should be recognized, however, that such description is not intended to limit the embodiments described to specific implementations, configurations, etc. Nor do the descriptions necessarily provide complete descriptions of the embodiments. As such, certain aspects, features, components, etc., may be omitted from the description of the various embodiments for ease of explanation.

According to various embodiments described herein, a new quantification model that can be used to accommodate for volumetric variation and recover the quantification result at high precision is provided. Monte Carlo simulations are used to demonstrate the efficacy of the proposed model.

### INTRODUCTION

In general, a digital PCR method distributes target molecules into a large number of partitions such that each partition gets a number of molecules (0, 1, 2, etc.) theoretically following a Poisson distribution. Performing PCR on these partitions results in amplification being detected (positives) in those partitions containing one or more target molecules and no amplification being detected (negatives) in those partitions containing zero target molecules. As positives may contain more than one copy of the target molecule, a simple summing of the number of positives will not yield the correct number of target molecules present across the partitions. Currently, Poisson statistics are widely employed to estimate the total number of target molecules within the interrogated sample. For a detailed review of standard digital PCR modeling and characteristics, refer to [Majumdar, N., Wessel, T., Marks, J., “Digital PCR Modeling for Maximal Sensitivity, Dynamic Range and Measurement Precision,” in PLOS One, 2015, pp. 1-17.]

As described below, according to various embodiments described herein, partitions may include, but are not limited to, through-holes, sample retainment regions, wells, indentations, spots, cavities, reaction chambers, and droplets for example.

Furthermore, as used herein, amplification may include thermal cycling, isothermal amplification, thermal convention, infrared mediated thermal cycling, or helicase dependent amplification, for example.

According to various embodiments, detection of a target nucleic acid may be, but is not limited to, fluorescence detection, detection of positive or negative ions, pH detection, voltage detection, or current detection, alone or in combination, for example.

### Implications of a Non-Mono-Dispersed Partition Size for Poisson Based Quantification

Poisson statistics are founded on the notion that the probability of any event occurring within a bounded volume depends only upon the size of the volume itself. Digital PCR systems, by their very nature, divide interrogated samples into a set of smaller partitions. It is common practice to make the assumption of mono-disperse partitioning to allow for the simplification of assigning a common probability of acquiring any given target molecule to each of the partitions. Mono-disperse means all of the partitions are identically sized.

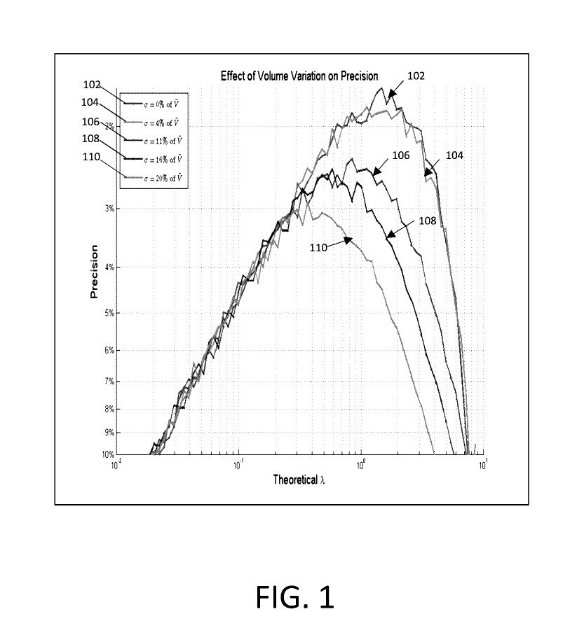

The effect of volume variation among reaction chambers on estimating concentration was investigated with Monte Carlo simulations. In this simulation, the average number of molecules in a partition λ is proportional to the volume of the partition. A normal distribution of volume variation is assumed with the standard deviation taken as a percentage of the mean volume. Data traces 110, 108, 106, 104, and 102 show 0%, 4%, 11%, 16%, and 20% of the mean volumes, respectively. FIG. 1 shows that volume variation impacts higher concentration more significantly than lower concentration. The process will underestimate at higher concentrations proportional to the degree of variation.

FIG. 1 shows that the effect of volume variation on precision is significant at higher concentrations. Volume variability is simulated by assuming a normal distribution of well volumes with the standard deviation taken as a percentage of the mean well volume. Volumes of 865 pl for 10,000 partitions were used in the simulation. Note that concentration at peak precision moves toward lower concentrations (increasing negative percentage) as volume variability increases.

### Beyond the Poisson Model for Digital PCR Systems

For the Poisson model, the mean number of molecules per partition (λ) is assumed to be constant. For the volume variation method according to various embodiments described herein, assume that the number of average molecules in each reaction λ(v) is proportional to the volume v caught in it as given in Equation (1). The constant of proportionality C is defined as the concentration of the target molecules, which is the quantity of interest in this measurement exercise.

λ(v)=Cv (1)

Consider the joint probability distribution of a partition being negative and constraining the volume V. One can apply Bayes' theorem [Bayes, Thomas, and Price, Richard, “An Essay towards solving a Problem in the Doctrine of Chance. By the late Rev. Mr. Bayes, communicated by Mr. Price, in a letter to John Canton, A. M. F. R. S,” in Philosophical Transactions of the Royal Society of London 53 (0): pp. 370-418.] as given in equation (2) to arrive at the joint probability distribution of a partition being negative (i.e. containing no molecules) and constraining a volume v.

P(neg,v)=P(neg|v)P(v) (2)

The probability of a partition to not receive any template is given by equation (3) according to the standard Poisson distribution, evaluated at number of molecules=0.

$$P ( neg \vert v ) = {e}^{- Cv \frac{{( Cv )}^{n}}{n !}} {\vert}_{n = 0} = {e}^{- Cv} \qquad ( 3 )$$

Assume the partition sizes follow a truncated normal distribution [symbol] (v₀, σ) with the parameters v₀ and σ as given by equation 4. A truncated distribution is assumed, as volumes cannot take negative values

$$P ( v ) = K {e}^{- \frac{- {( v - {v}_{0} )}^{2}}{2 {σ}^{2}}} \qquad ( 4 )$$

(K is a constant of proportionality, whose expression is derived in Section A, and is given by

$$K = \frac{1}{σ \sqrt{\frac{π}{2} erfc ( - \frac{{v}_{0}}{\sqrt{2} σ} )}} . )$$

Using equations (3) and (4) in (2), the joint probability of a negative partition to constrain a volume v is given by equation (5).

$$P ( neg , v ) = K {e}^{- Cv} {e}^{- \frac{- {( v - {v}_{0} )}^{2}}{2 {σ}^{2}}} \qquad ( 5 )$$

Now, the variable v may be integrated out as in equation (6).

P(neg)=∫₀^{∞}P(neg,v)dv (6)

The final expression for P(neg) is given as in equation (7).

$$P ( neg ) = \frac{erfc [ - \frac{1}{\sqrt{2}} ( \frac{{v}_{0}}{σ} - C σ ) ]}{erfc [ - \frac{1}{\sqrt{2}} ( \frac{{v}_{0}}{σ} ) ]} {e}^{- {Cv}_{0} + \frac{1}{2} {σ}^{2} {C}^{2}} \qquad ( 7 )$$

Equation (7) needs to be solved implicitly in order to evaluate concentration C. The derivation of the expression for P(neg) in equation (7) is given in the supporting documents, Section A. The results from this model are referred to as “Variable λ Full Fidelity” or the “Variable λ” model in subsequent sections.

Alternately, an approximation may also be used, yielding a direct closed form expression for concentration C, as given in equation (8). The derivation for equation (8) is given in the supporting documents, Section B. The results from this model are referred to as “Variable λ Approximation” model in subsequent sections.

$$C = \frac{{v}_{o} - \sqrt{{v}_{0}^{2} + 2 {σ}^{2} ln P ( neg )}}{{σ}^{2}} \qquad ( 8 )$$

The confidence intervals around the quantification by this new model are created by assuming that the variable log C is normally distributed. Equation (9) describes how this formalism is valid for computing confidence intervals.

$$\begin{matrix}CI = log C \pm z σ & ( 9 ) \\ where σ = 1 / \sqrt{nw} & ( 10 ) \\ and w = \frac{1}{P ( 1 - P )} {{C}^{2} ( \frac{d P}{d C} )}^{2} & ( 11 )\end{matrix}$$

and z is the z-score associated with the desired confidence interval.

Now, using Equation (7) above, for P

$$P ( neg ) = \frac{erfc [ - \frac{1}{\sqrt{2}} ( \frac{{v}_{0}}{σ} - C σ ) ]}{erfc [ - \frac{1}{\sqrt{2}} ( \frac{{v}_{0}}{σ} ) ]} {e}^{- {Cv}_{0} + \frac{1}{2} {σ}^{2} {C}^{2}} \\ As erfc ( z ) = {\int }_{z}^{∞} \frac{2}{\sqrt{π}} {e}^{- {t}^{2}} d t \\ So \frac{d }{d z} erfc ( z ) = - \frac{2}{\sqrt{π}} {e}^{- {z}^{2}} \\ Hence , \frac{d }{d C} erfc [ - \frac{1}{2} ( \frac{{v}_{0}}{σ} - C σ ) ] = - \frac{2}{\sqrt{π}} {e}^{- {( \frac{{v}_{0}}{σ} - C σ )}^{2}} \frac{σ}{\sqrt{2}} \qquad ( 7 )$$

Further manipulations will show that:

$$\frac{d P}{d C} = P ( neg ) [ ( - {v}_{0} + {σ}^{2} C ) - σ \sqrt{\frac{2}{π}} \frac{{e}^{- \frac{1}{2} {( \frac{{v}_{0}}{σ} - C σ )}^{2}}}{erfc ( - \frac{1}{2} ( \frac{{v}_{0}}{σ} - C σ ) )} ] \qquad ( 12 )$$

Equation (12) is substituted in equation (11), which is substituted into equation (10) and back out to equation (9) to yield the requisite confidence intervals.

### Results

### Simulation Results: Impact on the Dynamic Range

Monte Carlo simulations were run to demonstrate the remedial effects of quantification using alternate modeling that accounts for non-uniform partition size for Poisson processes. These simulations assume that the average number of target nucleic acids in a partition is proportional to the volume of the partition. A normal distribution of volume variation is assumed with the standard deviation taken as a percentage of the mean volume. The steps of generating the simulation data are outlined in Section C.

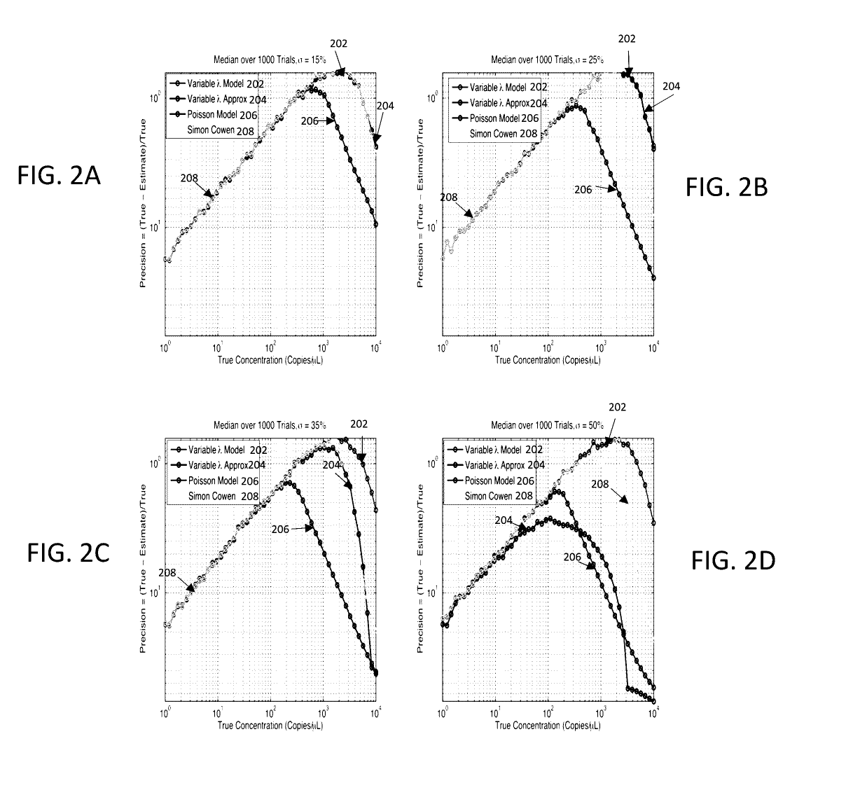

An alternate model accommodating partition size variation was proposed by Cowen [Huggett J., Cowen S., and Foy, C. Considerations for Digital PCR as an Accurate Molecular Diagnostic Tool² in Clinical Chemistry 61:1, 2015, pp. 79-88]. Results from the Poisson model, the Simon Cowen model, the variable λ approximation model and the variable λ full fidelity model and are compared in the set of FIG. 2: FIGS. 2A, 2B, 2C and 2D show increasingly higher partition size non-uniformities created by assuming a standard deviation of 15%, 25%, 35% and 50% of the mean volumes, respectively.

FIG. 2: Remedial Power of Partition Size Non-uniformity Sensitive Modeling of Poisson Processes on Precision Results from the Poisson model 206, the Simon Cowen model 208 and the two versions 202 and 204 of the currently proposed volume variable λ model are compared. The Poisson model 206 is consistently the worst at higher concentrations for all levels of variations under consideration. FIGS. 2B, 2C and 2D shows that at the higher levels of standard deviation=25%, 35% and 50%, the full fidelity volume variable λ model 202 demonstrates the best performance in recovering from errors in quantification introduced by non-uniform partition size.

FIG. 2A shows that at a 15% volume variation, the variable λ approx. model 204, the volume variable λ full fidelity model 202 and the Simon Cowen model 208 agree closely and all outperform Poisson model 206 consistently for concentrations above 400 copies/microliter. FIG. 2B shows that at a 25% volume variation, the volume variable λ approx. model 204, the volume variable λ full fidelity model 202 and Simon Cowen's model 208 agrees up to a concentration of approximately 1000 copies/microliter. Beyond this, the Simon Cowen model 208 and the two versions of the volume variable λ models 202 and 204 begin to diverge, with the volume variable λ models 202 and 204 demonstrating the superior performance. The Poisson model 206 is consistently the worst at all concentrations beyond 100 copies/microliter. FIGS. 2C and 2D continues to show that at the 35% and 50% volume variations respectively, the volume variable λ models 202 and 204 demonstrate the best performance in recovering from errors in quantification introduced by non-uniform partition size. At 50%, the volume variable λ approximate model 204 is no longer usable.

### CONCLUSIONS

Poisson based quantification is sensitive to partition size non-uniformity, particularly at the higher concentration limits. The impact of the non-uniformity is also felt in quantification of the rare target as the quantification of the wild target is impacted by it. Models that take this variation into account, as described in various embodiments described herein, can be harnessed to quantify accurately despite the variation. A major factor to the success in application of these models is in the correct assessment of the true levels of effective volume variation of the partition size. In array based systems, the partition size meaning the through-hole could be very uniform. Nevertheless, the loading volume may vary across the different through-holes because of other factors such as coating or reaction formulation. This effectively makes the partition from the Poisson modeling stand point more variable than the physical dimension of the through-hole. For the volume variation modeling, what is important is the volume of sample that is within the partition, which is the effective volume.

### Section A: Derivation of the Expression for the Probability of Negatives in the Variable λ Full Fidelity Model

We assumed that the partition sizes follow a truncated normal distribution [symbol] (v₀, σ) with a mean volume of v₀ and a standard deviation of σ as given by equation 8. A truncated distribution is assumed, as volumes cannot take negative values. To evaluate the constant factor K, we integrate the probability distribution function and equate it to 1.

$${\int }_{0}^{∞} P ( v ) d v = 1$$

$$Or , K {\int }_{0}^{∞} {e}^{- \frac{{( v - {v}_{0} )}^{2}}{2 {σ}^{2}}} d v = 1$$

$$Let {v}^{′} = \frac{( v - {v}_{0} )}{\sqrt{2} σ} ⇒ {dv}^{′} = \frac{dv}{\sqrt{2} σ}$$

$$Or , K {\int }_{0}^{∞} {e}^{- {( {v}^{′} )}^{2}} d {v}^{′} = 1$$

$$Or , K \cdot σ \cdot \sqrt{\frac{π}{2}} {erfc ( - \frac{{v}_{0}}{\sqrt{2} σ} )}^{- \frac{{v}_{0}}{\sqrt{2} σ}} = 1$$

$$Or , K = \frac{1}{σ \sqrt{\frac{π}{2}} erfc ( - \frac{{v}_{0}}{\sqrt{2} σ} )}$$

Note as

$${v}_{0} -> + ∞ , erfc -> 2 , ⇒ K = \frac{1}{σ \sqrt{2 π}} ,$$

and we obtain the well-known constant factor associated with Gaussian distributions between −∞ to +∞.

Now, using this K in equation 5, we obtain:

$$P ( neg , v ) = \frac{1}{σ \sqrt{\frac{π}{2}} erfc [ - \frac{1}{\sqrt{2}} ( \frac{{v}_{0}}{σ} )} {e}^{- Cv} {e}^{- \frac{{( v - {v}_{0} )}^{2}}{2 {σ}^{2}}}$$

And following on with the integration step as outlined in equation (6), we obtain:

$$\begin{matrix}\begin{matrix}P ( neg ) = {\int }_{0}^{∞} P ( neg , v ) d v \\ = \frac{1}{σ \sqrt{\frac{π}{2}} erfc [ - \frac{1}{\sqrt{2}} ( \frac{{v}_{0}}{σ} ) ]} {\int }_{0}^{∞} {e}^{- Cv} {e}^{- \frac{{( v - {v}_{0} )}^{2}}{2 {σ}^{2}}} d v \\ = \frac{1}{σ \sqrt{\frac{π}{2}} erfc [ - \frac{1}{\sqrt{2}} ( \frac{{v}_{0}}{σ} ) ]} I\end{matrix} \\ Where I = {\int }_{0}^{∞} {e}^{- Cv} {e}^{- \frac{{( v - {v}_{0} )}^{2}}{2 {σ}^{2}}} d v & ( i ) \\ \begin{matrix}I = {\int }_{0}^{∞} {e}^{- Cv} {e}^{- \frac{( {v}^{2} - 2 {vv}_{0} + {v}_{0}^{2} )}{2 {σ}^{2}}} d v \\ = {e}^{- \frac{{v}_{0}^{2}}{2 {σ}^{2}}} {\int }_{0}^{∞} {e}^{- Cv} {e}^{- \frac{( {v}^{2} - 2 {vv}_{0} )}{2 {σ}^{2}}} d v \\ = {e}^{- \frac{{v}_{0}^{2}}{2 {σ}^{2}}} {\int }_{0}^{∞} {e}^{- \frac{{v}^{2}}{2 {σ}^{2}}} {e}^{v ( \frac{{v}_{0}}{{σ}^{2}} - C )} d v \\ = {e}^{- \frac{{v}_{0}^{2}}{2 {σ}^{2}}} {\int }_{0}^{∞} {e}^{- {Av}^{2} + Bv} d v ,\end{matrix} \\ where A = \frac{1}{2 {σ}^{2}} , B = \frac{{v}_{0}}{{σ}^{2}} - C & ( ii ) \\ \begin{matrix}Let {I}^{′} = {\int }_{0}^{∞} {e}^{- {Av}^{2} + Bv} d v \\ = {\int }_{0}^{∞} {e}^{- A ( {v}^{2} - \frac{B}{A} v )} d v \\ = {\int }_{0}^{∞} {e}^{- A ( {v}^{2} - 2 v \frac{B}{2 A} + \frac{{B}^{2}}{4 {A}^{2}} - \frac{{B}^{2}}{4 {A}^{2}} )} d v \\ = {\int }_{0}^{∞} {e}^{- {A ( v - \frac{B}{2 A} )}^{2}} {e}^{\frac{{B}^{2}}{4 {A}^{2}}} d v \\ = {\int }_{0}^{∞} {e}^{- \frac{1}{2 {σ}^{2}} {( v - \frac{\frac{{v}_{0}}{{σ}^{2}} - C}{\frac{1}{{σ}^{2}}} )}^{2}} {e}^{\frac{{( \frac{{v}_{0}}{{σ}^{2}} - C )}^{2}}{4 \frac{1}{2 {σ}^{2}}}} d v \\ = {\int }_{0}^{∞} {e}^{- \frac{1}{2 {σ}^{2}} {( v - {σ}^{2} ( \frac{{v}_{0}}{{σ}^{2}} - C ) )}^{2}} {e}^{\frac{{σ}^{2}}{2} {( \frac{{v}_{0}}{{σ}^{2}} - C )}^{2}} d v \\ = {\int }_{0}^{∞} {e}^{- \frac{1}{2 {σ}^{2}} {( v - {v}_{0} + C {σ}^{2} )}^{2}} {e}^{\frac{{σ}^{2}}{2} ( \frac{{v}_{0}^{2}}{{σ}^{4}} - 2 C \frac{{v}_{0}}{{σ}^{2}} + {C}^{2} )} d v \\ = {\int }_{0}^{∞} {e}^{- \frac{1}{2 {σ}^{2}} {( v - {v}_{0} + C {σ}^{2} )}^{2}} {e}^{\frac{{v}_{0}^{2}}{2 {σ}^{2}}} {e}^{\frac{{σ}^{2} {C}^{2}}{2}} d v\end{matrix} & ( iii )\end{matrix}$$

Using (iii) in (ii), we obtain:

$$\begin{matrix}I = {e}^{- {Cv}_{0}} {e}^{\frac{{σ}^{2} {C}^{2}}{2}} {\int }_{0}^{∞} {e}^{- \frac{1}{2 {σ}^{2}} {( v - {v}_{0} + C {σ}^{2} )}^{2}} d v \\ Let {I}^{″} = {\int }_{0}^{∞} {e}^{- \frac{1}{2 {σ}^{2}} ( v - {v}_{0} + C {σ}^{2} )} d {v}^{′} \\ Let {v}^{″} = \frac{1}{\sqrt{2} σ} ( v - ( {v}_{0} - C {σ}^{2} ) ) & ( iv ) \\ \begin{matrix}Or , {I}^{″} = {\int }_{- \frac{( {v}_{0} - C {σ}^{2} )}{\sqrt{2} σ}}^{∞} {e}^{- {v}^{″2}} \sqrt{2} σ d {v}^{″} \\ = \sqrt{2} σ \frac{\sqrt{π}}{2} \frac{2}{\sqrt{π}} {\int }_{- \frac{( {v}_{0} - C {σ}^{2} )}{\sqrt{2} σ}}^{∞} {e}^{- {v}^{″2}} d {v}^{″} \\ = \sqrt{2} σ \frac{\sqrt{π}}{2} erfc ( - \frac{( {v}_{0} - C {σ}^{2} )}{\sqrt{2} σ} )\end{matrix} & ( v )\end{matrix}$$

Using (iv) and (v) in (i):

$$P ( neg ) = \frac{1}{σ \sqrt{\frac{π}{2}} erfc [ - \frac{1}{\sqrt{2}} ( \frac{{v}_{0}}{σ} ) ]} \sqrt{2} σ \frac{\sqrt{π}}{2} erfc [ - \frac{1}{\sqrt{2}} ( \frac{{v}_{0}}{σ} - C σ ) ] {e}^{- {Cv}_{0} + \frac{1}{2} {σ}^{2} {C}^{2}} = \frac{erfc [ - \frac{1}{\sqrt{2}} ( \frac{{v}_{0}}{σ} - C σ ) ]}{erfc [ - \frac{1}{\sqrt{2}} ( \frac{{v}_{0}}{σ} ) ]} {e}^{- {Cv}_{0} + \frac{1}{2} {σ}^{2} {C}^{2}}$$

### Section B: Derivation of the Expression for the Probability of Negatives in the Variable λ Approximation Model

If we assume that the partition sizes follow a normal distribution [symbol] (v₀, σ) with a mean volume of v₀ and a standard deviation of σ, then, by definition P(v) given in equation 4 becomes:

$$P ( v ) = {e}^{- \frac{- {( v - {v}_{0} )}^{2}}{2 {σ}^{2}}} \frac{1}{σ \sqrt{2 π}}$$

Substituting this P(v) in equation 5,

$$P ( neg , v ) = {e}^{- μ v} {e}^{- \frac{- {( v - {v}_{0} )}^{2}}{2 {σ}^{2}}} \frac{1}{σ \sqrt{2 π}}$$

Now, integrating the variable v out as follows:

$$\begin{matrix}P ( neg ) = {\int }_{- ∞}^{∞} P ( neg , v ) d v \\ = \frac{1}{σ \sqrt{2 π}} {\int }_{- ∞}^{∞} {e}^{- Cv} {e}^{- \frac{- {( v - {v}_{0} )}^{2}}{2 {σ}^{2}}} d v \\ = \frac{1}{σ \sqrt{2 π}} {\int }_{- ∞}^{∞} {e}^{- Cv} {e}^{- \frac{[ {v}^{2} - 2 {vv}_{0} + {v}_{0}^{2} ]}{2 {σ}^{2}}} d v \\ = \frac{1}{σ \sqrt{2 π}} {e}^{- \frac{{v}_{0}^{2}}{2 {σ}^{2}}} {\int }_{- ∞}^{∞} {e}^{- Cv} {e}^{- \frac{{v}^{2}}{2 {σ}^{2}}} {e}^{\frac{{vv}_{0}}{{σ}^{2}}} d v \\ = \frac{1}{σ \sqrt{2 π}} {e}^{- \frac{{v}_{0}^{2}}{2 {σ}^{2}}} {\int }_{- ∞}^{∞} {e}^{- \frac{{v}^{2}}{2 {σ}^{2}}} {e}^{v ( \frac{{v}_{0}}{{σ}^{2}} - C )} d v\end{matrix} \qquad ( i )$$

It is known that:

$${\int }_{- ∞}^{∞} {e}^{{ax}^{2} + bx} d x = \sqrt{π / - a} {e}^{- {b}^{2} / 4 a} .$$

So (i) becomes:

$$= \frac{1}{σ \sqrt{2 π}} {e}^{- \frac{{v}_{0}^{2}}{2 {σ}^{2}}} \sqrt{π ( 2 {σ}^{2} )} {e}^{- \frac{{( \frac{{v}_{0}}{{σ}^{2}} - C )}^{2}}{4 ( - \frac{1}{2 {σ}^{2}} )}} \\ = \frac{1}{σ \sqrt{2 π}} \frac{\sqrt{π ( 2 {σ}^{2} )} {e}^{( \frac{{v}_{0}^{2}}{{σ}^{4}} + {C}^{2} - \frac{\overline{2} {Cv}_{0}}{{σ}^{2}} ) \frac{{σ}^{2}}{2}}}{{e}^{\frac{{v}_{0}^{2}}{2 {σ}^{2}}}} \\ = \frac{1}{\overline{σ \sqrt{2 π}}} \frac{\overline{\sqrt{π ( 2 {σ}^{\overline{2}} )}} {e}^{( \frac{{v}_{0}^{2}}{{σ}^{4}} + {C}^{2} - \frac{\overline{2} {Cv}_{0}}{{σ}^{2}} ) \frac{{σ}^{2}}{2}}}{{e}^{\frac{{v}_{0}^{2}}{2 {σ}^{2}}}} \\ = \frac{\frac{\overline{{v}_{\overline{0}}^{\overline{2}}}}{\overline{{e}^{\overline{2}} {σ}^{\overline{2}}}} {e}^{( \frac{{C}^{2} {σ}^{2}}{2} - \frac{\overline{2} {Cv}_{0} \overline{{σ}^{\overline{2}}}}{\overline{{σ}^{\overline{2}}} \overline{2}} )}}{\frac{\overline{{v}_{\overline{0}}^{\overline{2}}}}{\overline{{e}^{\overline{2}} {σ}^{\overline{2}}}}} \\ = {e}^{( \frac{1}{2} {σ}^{2} {C}^{2} - {Cv}_{0} )} \\ Or , P ( neg ) = {e}^{( \frac{1}{2} {σ}^{2} {C}^{2} - {Cv}_{0} )} \qquad ( ii )$$

Solve for concentration C as follows:

$$P ( neg ) = exp ( \frac{1}{2} {σ}^{2} {C}^{2} - {Cv}_{0} ) \\ ln P ( neg ) = \frac{1}{2} {σ}^{2} {C}^{2} - {Cv}_{0} \\ \frac{1}{2} {σ}^{2} {C}^{2} - {Cv}_{0} - ln P ( neg ) = 0 \\ C = \frac{{v}_{0} \pm \sqrt{{v}_{0}^{2} + 4 \frac{1}{2} {σ}^{2} ln P ( neg )}}{2 \frac{1}{2} {σ}^{2}} \\ C = \frac{{v}_{0} \pm \sqrt{{v}_{0}^{2} + 2 {σ}^{2} ln P ( neg )}}{{σ}^{2}} \qquad ( iii )$$

Note that C can assume two values, but the value with the negative sign in front of the square root sign in the numerator in (iii) is used as it can be shown that it agrees in the small σ limit with the solution from using a Poisson model for the case where variability is assumed to be 0.

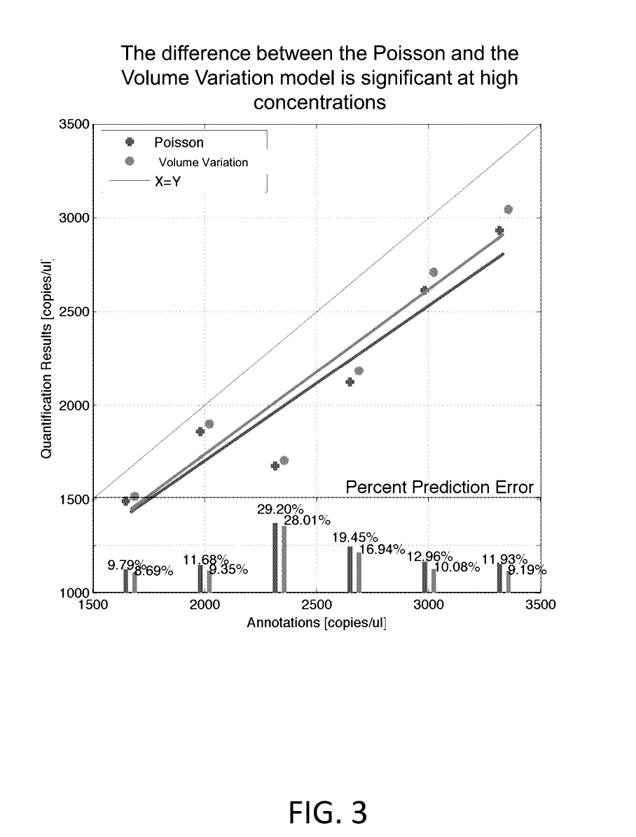

With reference to FIG. 3, the improvement of the results using a volume variation model over the traditional Poisson model is illustrated. In this graph, a set of digital PCR experiments was run using ERM Plasmid samples and the BCR-ABL1 Taqman assay using standard protocol prescribed for the QuantStudio 3D Digital PCR system. A QuantStudio 3D chip contains a plurality of partitions. Up to 6 replicate chips were run at each concentration interrogated in this experiment. Only chips that passed visual quality inspection were included in the analysis. Chips were filtered out if they showed artifacts such as bridging. The positive and negative counts from the chips were used with both the Poisson and the volume variation models to generate a quantification result. The mean result from each model is reported in FIG. 3. The +− one standard deviation around this mean value is also shown in the figure. The bottom section shows bar graphs representing the percent prediction error based upon annotations of what concentration was run on the chips. The prediction error is consistently higher for the Poisson model showing the better performance of the Poisson Plus modeling.

Those skilled in the art will recognize that the operations of the various embodiments may be implemented using hardware, software, firmware, or combinations thereof, as appropriate. For example, some processes can be carried out using processors or other digital circuitry under the control of software, firmware, or hard-wired logic. (The term “logic” herein refers to fixed hardware, programmable logic and/or an appropriate combination thereof, as would be recognized by one skilled in the art to carry out the recited functions.) Software and firmware can be stored on non-transitory computer-readable media. Some other processes can be implemented using analog circuitry, as is well known to one of ordinary skill in the art. Additionally, memory or other storage, as well as communication components, may be employed in embodiments of the present teachings.

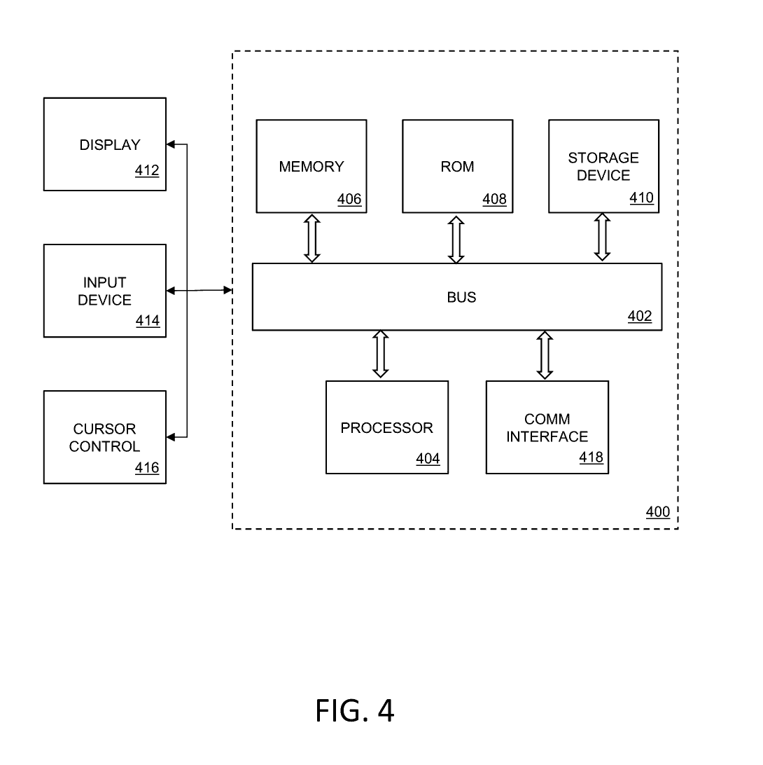

FIG. 4 is a block diagram that illustrates a computer system 400 that can be employed to carry out processing functionality, and to implement various components or subsystems of the systems described herein according to various embodiments. For example, system 400 can comprise all or apportion of devices 540, client devices, 502, 512, or 530, servers 522, etc. Computing system 400 can include one or more processors, such as a processor 404. Processor 404 can be implemented using a general or special purpose processing engine such as, for example, a microprocessor, controller or other control logic. In this example, processor 404 is connected to a bus 402 or other communication medium.

Further, it should be appreciated that a computing system 400 of FIG. 4 can be embodied in any of a number of forms, such as a rack-mounted computer, mainframe, supercomputer, server, client, a desktop computer, a laptop computer, a tablet computer, hand-held computing device (e.g., PDA, cell phone, smart phone, palmtop, etc.), cluster grid, netbook, embedded systems, or any other type of special or general purpose computing device as may be desirable or appropriate for a given application or environment. Additionally, a computing system 400 can include a conventional network system including a client/server environment and one or more database servers, or integration with LIS/LIMS infrastructure. A number of conventional network systems, including a local area network (LAN) or a wide area network (WAN), and including wireless and/or wired components, are known in the art. Additionally, client/server environments, database servers, and networks are well documented in the art. According to various embodiments described herein, computing system 400 may be configured to connect to one or more servers in a distributed network. Computing system 400 may receive information or updates from the distributed network. Computing system 400 may also transmit information to be stored within the distributed network that may be accessed by other clients connected to the distributed network.

Computing system 400 may include bus 402 or other communication mechanism for communicating information, and processor 404 coupled with bus 402 for processing information.

Computing system 400 also includes a memory 406, which can be a random access memory (RAM) or other dynamic memory, coupled to bus 402 for storing instructions to be executed by processor 404. Memory 406 also may be used for storing temporary variables or other intermediate information during execution of instructions to be executed by processor 404. Computing system 400 further includes a read only memory (ROM) 408 or other static storage device coupled to bus 402 for storing static information and instructions for processor 404.

Computing system 400 may also include a storage device 410, such as a magnetic disk, optical disk, or solid state drive (SSD) is provided and coupled to bus 402 for storing information and instructions. Storage device 410 may include a media drive and a removable storage interface. A media drive may include a drive or other mechanism to support fixed or removable storage media, such as a hard disk drive, a floppy disk drive, a magnetic tape drive, an optical disk drive, a CD or DVD drive (R or RW), flash drive, or other removable or fixed media drive. As these examples illustrate, the storage media may include a computer-readable storage medium having stored therein particular computer software, instructions, or data.

In alternative embodiments, storage device 410 may include other similar instrumentalities for allowing computer programs or other instructions or data to be loaded into computing system 400. Such instrumentalities may include, for example, a removable storage unit and an interface, such as a program cartridge and cartridge interface, a removable memory (for example, a flash memory or other removable memory module) and memory slot, and other removable storage units and interfaces that allow software and data to be transferred from the storage device 410 to computing system 400.

Computing system 400 can also include a communications interface 418. Communications interface 418 can be used to allow software and data to be transferred between computing system 400 and external devices. Examples of communications interface 418 can include a modem, a network interface (such as an Ethernet or other NIC card), a communications port (such as for example, a USB port, a RS-232C serial port), a PCMCIA slot and card, Bluetooth, etc. Software and data transferred via communications interface 418 are in the form of signals which can be electronic, electromagnetic, and optical or other signals capable of being received by communications interface 418. These signals may be transmitted and received by communications interface 418 via a channel such as a wireless medium, wire or cable, fiber optics, or other communications medium. Some examples of a channel include a phone line, a cellular phone link, an RF link, a network interface, a local or wide area network, and other communications channels.

Computing system 400 may be coupled via bus 402 to a display 412, such as a cathode ray tube (CRT) or liquid crystal display (LCD), for displaying information to a computer user. An input device 414, including alphanumeric and other keys, is coupled to bus 402 for communicating information and command selections to processor 404, for example. An input device may also be a display, such as an LCD display, configured with touchscreen input capabilities. Another type of user input device is cursor control 416, such as a mouse, a trackball or cursor direction keys for communicating direction information and command selections to processor 404 and for controlling cursor movement on display 412. This input device typically has two degrees of freedom in two axes, a first axis (e.g., x) and a second axis (e.g., y), that allows the device to specify positions in a plane. A computing system 400 provides data processing and provides a level of confidence for such data. Consistent with certain implementations of embodiments of the present teachings, data processing and confidence values are provided by computing system 400 in response to processor 404 executing one or more sequences of one or more instructions contained in memory 406. Such instructions may be read into memory 406 from another computer-readable medium, such as storage device 410. Execution of the sequences of instructions contained in memory 406 causes processor 404 to perform the process states described herein. Alternatively hard-wired circuitry may be used in place of or in combination with software instructions to implement embodiments of the present teachings. Thus implementations of embodiments of the present teachings are not limited to any specific combination of hardware circuitry and software.

The term “computer-readable medium” and “computer program product” as used herein generally refers to any media that is involved in providing one or more sequences or one or more instructions to processor 404 for execution. Such instructions, generally referred to as “computer program code” (which may be grouped in the form of computer programs or other groupings), when executed, enable the computing system 400 to perform features or functions of embodiments of the present embodiments described herein. These and other forms of non-transitory computer-readable media may take many forms, including but not limited to, non-volatile media, volatile media, and transmission media. Non-volatile media includes, for example, solid state, optical or magnetic disks, such as storage device 410. Volatile media includes dynamic memory, such as memory 406. Transmission media includes coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, a carrier wave as described hereinafter, or any other medium from which a computer can read.

Various forms of computer readable media may be involved in carrying one or more sequences of one or more instructions to processor 404 for execution. For example, the instructions may initially be carried on magnetic disk of a remote computer. The remote computer can load the instructions into its dynamic memory and send the instructions over a telephone line using a modem. A modem local to computing system 400 can receive the data on the telephone line and use an infra-red transmitter to convert the data to an infra-red signal. An infra-red detector coupled to bus 402 can receive the data carried in the infra-red signal and place the data on bus 402. Bus 402 carries the data to memory 406, from which processor 404 retrieves and executes the instructions. The instructions received by memory 406 may optionally be stored on storage device 410 either before or after execution by processor 404.

It will be appreciated that, for clarity purposes, the above description has described embodiments with reference to different functional units and processors. However, it will be apparent that any suitable distribution of functionality between different functional units, processors or domains may be used without detracting from the embodiments of the present teachings. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

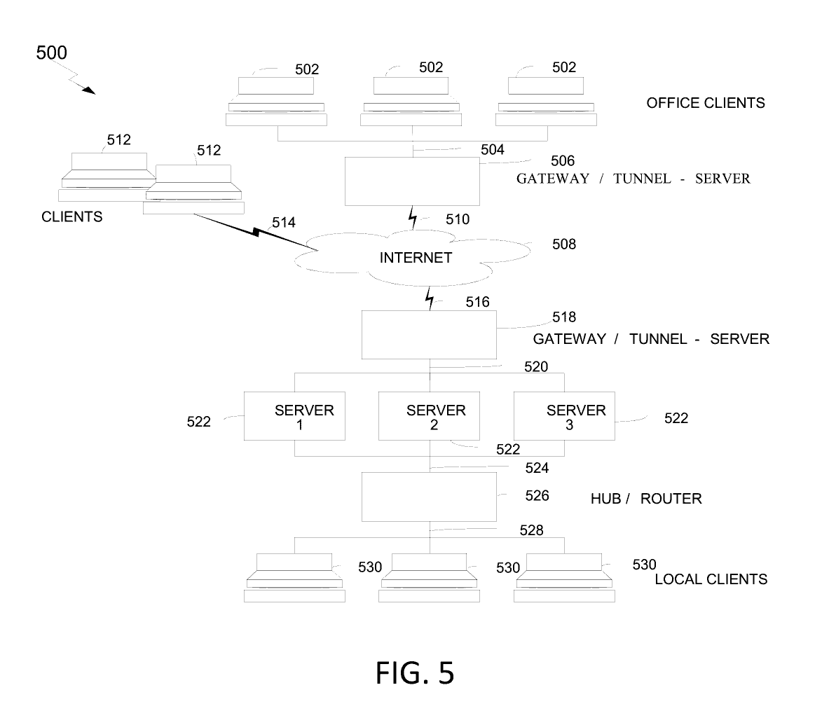

FIG. 5 is a diagram illustrating an example system 500 configured in accordance with one example embodiment. In system 500, one or more servers 522 can be configured to run the analysis applications for analyzing data sets produced by one or more devices or modalities 540. The data included in the data sets can be stored in one or more storage devices 550. Once the data sets have been uploaded to servers 522, then a plurality of applications running on servers 522 can be used to manipulate, analyze and visualize the data sets from anywhere. For example, local client devices 530 can be used to access servers 522, e.g., through a hub or router 526. At the same time, the data can be accessed remotely through remote clients devices 502, which are interfaced with servers 522, e.g., via a gateway/hub/tunnel-server/etc. 510, which is itself connected to the internet 508 via some internet service provider (ISP) connection 510, or remote client servers 512, which are interfaced with servers 522, e.g., via the internet 508 and via an ISP connection 514.

It should also be noted that devices 540 can be directly interfaced with servers 522, e.g., through the internet. In such embodiments, the collection application and functionality can reside on servers 522, on devices 540, or both. In other embodiments, devices 540 can be interfaced with client devices 502 or 512. In such embodiments, the collection application or functionality can be included on client devices 502 or 512, devices 540, or both.

Client devices 502, 512, and 530 can be any kind of computing device that can be used to access servers 522. As such, these devices can be laptop, desktop, or palmtop computers, terminals, mobile computing devices such as smartphones or tablets, etc. Servers 522 can comprise one or more processors, servers, routers, co-processors, user interfaces, etc., whether co-located or located in different locations. In short, servers 522 can comprise all of the resources, both hardware and software, needed to perform the functions described herein. A more detailed description of a computer system and the resources that can be used to implement the components illustrated in FIG. 5 is described below with respect to FIG. 4.

Although various embodiments have been described with respect to certain exemplary embodiments, examples, and applications, it will be apparent to those skilled in the art that various modifications and changes may be made without departing from the present teachings.

What is claimed is:

1. A method for performing digital polymerase chain reaction (dPCR), the method comprising:
partitioning a biological sample volume including a plurality of target nucleic acids into a plurality of partitions, wherein at least one partition includes at least one target nucleic acid;
determining a model for volume variation of the plurality of partitions;
determining a number of partitions including at least one target nucleic acid; and
generating a concentration of target nucleic acids in the biological sample based on the model for volume variation and the fraction of partitions including at least one target nucleic acid.

2. The method of claim 1, wherein the concentration of target nucleic acids in the biological sample is determined by using the equation:
$$C = \frac{{v}_{0} - \sqrt{{v}_{0}^{2} + 2 {σ}^{2} ln P ( neg )}}{{σ}^{2}} .$$

3. The method of claim 1, wherein the concentration of target nucleic acids in the biological sample is determined by using the equation:
$$P ( neg ) = \frac{erfc [ - \frac{1}{\sqrt{2}} ( \frac{{v}_{0}}{σ} - C σ ) ]}{erfc [ - \frac{1}{\sqrt{2}} ( \frac{{v}_{0}}{σ} ) ]} {e}^{- {Cv}_{0} + \frac{1}{2} {σ}^{2} {C}^{2}}$$

4. The method of claim 1, further comprising:
amplifying the target nucleic acids to determine the number of partitions including at least one target nucleic acid.

5. The method of claim 1, wherein the model for volume variation is:
$$P ( neg ) = \frac{erfc [ - \frac{1}{\sqrt{2}} ( \frac{{v}_{0}}{σ} - C σ ) ]}{erfc [ - \frac{1}{\sqrt{2}} ( \frac{{v}_{0}}{σ} ) ]} {e}^{- {Cv}_{0} + \frac{1}{2} {σ}^{2} {C}^{2}}$$

6. The method of claim 1, wherein the model for volume variation is:
P(neg)=exp(½σ²C²−Cv₀)

7. The method of claim 1, wherein the plurality of partitions is a plurality of reaction sites.

8. The method of claim 1, wherein the plurality of partitions is a plurality of throughholes.

9. The method of claim 1, wherein the plurality of partitions is a plurality of droplets.

10. A system for performing digital polymerase chain reaction (dPCR), the system comprising:
a device configured to partition a biological sample volume including a plurality of target nucleic acids into a plurality of partitions, wherein at least one partition includes at least one target nucleic acid;
a memory; and
a processor configured to:
determine a number of partitions including at least one target nucleic acid; and
generate a concentration of target nucleic acids in the biological sample based on a model for volume variation and the fraction of partitions including at least one target nucleic acid.

11. The system of claim 10, wherein the concentration of target nucleic acids in the biological sample is determined by using the equation:
$$C = \frac{{v}_{0} - \sqrt{{v}_{0}^{2} + 2 {σ}^{2} ln P ( neg )}}{{σ}^{2}} .$$

12. The system of claim 10, wherein the concentration of target nucleic acids in the biological sample is determined by using the equation:
$$P ( neg ) = \frac{erfc [ - \frac{1}{\sqrt{2}} ( \frac{{v}_{0}}{σ} - C σ ) ]}{erfc [ - \frac{1}{\sqrt{2}} ( \frac{{v}_{0}}{σ} ) ]} {e}^{- {Cv}_{0} + \frac{1}{2} {σ}^{2} {C}^{2}}$$

13. The system of claim 10, further comprising:
an amplification apparatus configured to amplify the target nucleic acids to determine the number of partitions including at least one target nucleic acid.

14. The system of claim 10, wherein the model for volume variation is:
$$P ( neg ) = \frac{erfc [ - \frac{1}{\sqrt{2}} ( \frac{{v}_{0}}{σ} - C σ ) ]}{erfc [ - \frac{1}{\sqrt{2}} ( \frac{{v}_{0}}{σ} ) ]} {e}^{- {Cv}_{0} + \frac{1}{2} {σ}^{2} {C}^{2}}$$

15. The system of claim 10, wherein the model for volume variation is:
P(neg)=exp(½σ²C²−Cv₀)

16. The system of claim 10, wherein the plurality of partitions is a plurality of reaction sites.

17. The system of claim 10, wherein the plurality of partitions is a plurality of throughholes.

18. The system of claim 10, wherein the plurality of partitions is a plurality of droplets.
